(19) United States
(12) Patent Application Publication
(10) Pub. No.: US 2011/0305731 A1
(43) Pub. Date: Dec. 15, 2011

(54) PHARMACEUTICAL FORMULATION BETWEEN JASMONATES AND IT DERIVATIVES AND NANOCARRIES OR MICROCARRIES

(76) Inventor: Jose Emilio Fehr Pereira Lopes, Sao Carlos (BR)

(21) Appl. No.: 13/054,110

(22) PCT Filed: May 28, 2009
(86) PCT No.: PCT/BR2009/000151

§ 371 Date: Jan. 14, 2011

(30) Foreign Application Priority Data
Jul. 15, 2008 (BR) PI0804172-5

(51) Int. Cl. A61K 31/19; A61P 31/00; A61P 31/10; A61P 29/00; A61P 35/00; A61K 9/00; A61P 31/12; A61P 31/18; A61P 31/04; A61P 25/00; A61P 25/24; A61K 31/215; A61P 35/04

(52) U.S. Cl. 424/400; 514/573; 514/530

(57) ABSTRACT

The present invention refers to pharmaceutical compositions comprising a jasmonic acid derivative and a carrier, preferably cyclodextrin or a polyamidoamine. The compositions may be used for treating cancer.

### FIELD OF INVENTION

The present invention remains formulations which jasmonates family compounds (CA 2630666) in combination with several compounds that have capacibility to form nano or micro encapsulation systems. Jasmonates Family compounds are defined as the compounds with cyclopentanone type structures; it is a group of plant hormones which helps to regulate plant growth and development. Jasmonates include jasmonic acid and its esters, as well many others derivates such as an example: methyl jasmonate (MeJa). Like the related prostaglandin hormones found in mammals, the jasmonates are cyclopentanone derivatives, which are derived biosynthetically from fatty acids. They are biosynthesized from linolenic acid by the octadecanoid pathway with the cyclopentanone ring. The present inventions, also includes the Jasmonates with artificially, or not, modified formulated structures with substitutions or inclusions of an amine molecule, and or amide molecule, or any other substance to increase the jasmonates effects, such as the transformation of the cyclopentanone ring into a cyclopentenone ring, with inclusions, and or substitutions, and or conjugations, and or additions, and or reductions, and or any other chemical process, and or not, with any other components, of any type, inside of a nanocarrier or microcarrier, as a single molecule, and or a mixture molecule compound. Nano carries or micro carries are compounds that can be use to forms inclusion compounds. For example, becomes with the host family of cyclodextrins (CDs), more specifically the native CDs, α-, β- and γ-CD. Alternatively, we consider that it is possible to all systems that can make inclusions compounds as an opportune alternative for the CDs. Define in these terms a series of hosts molecules, and or particles, and or aggregates as nanocarrier in general such as polymers, and or alternatively polymers, and or co-polymers, and or liposome, and or dendrimers, and or metallic nano spheres, and or mixed polymers, and or biopolymers, and or carbon structures carriers, and or silica structure carriers, and or silicon structures carriers, and or injectable micro and or nanocarriers, and or nanocarriers achieve tumor-selective accumulation through the enhanced permeability and retention effects and targeting molecules such as antibodies, peptides, ligands, or nucleic acids attached to the nanocarriers further enhance their recognition and internalization by the target tissues, and or nanocarriers stimuli-activate in the extracellular environment and or intracellular environment, and or nanosuspentions, and or nano tubes, and or nano wires, and or cationic SLN carriers, and or gelatin NPs carriers, and or PLGA NPs, and or PLGA nanospheres, and or hydrogel NPs structure carriers, and or CPP NPs structure carriers, and or Polymeric micelles as known as immunomicelles, and or functionalized NPs, and or nano crystals structures carriers. In general, that called phenomena, can be characterized by the example illustration reaction of formation as scheme bellow:

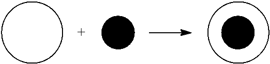

Same general reaction corresponds to the equation 3 at page 7. The compounds formed with these formulations are characterized as efficients systems to delivery Jasmonates family members and derivate to act macroscopic at specific target cells aim. These target cells are, or not, characterized to be cancer cells or any other site. These inclusion compounds can act with efficient performances with a significant reduce of the toxicity of jasmonates family members and derivate and chemical stabilization of the molecular structure of jasmonates family members and derivate from hydrolyses, and or oxidation, as example, and any other reaction (EP 1814894). These formulations present here are involved with the inclusion phenomena, the principle of host and guest interaction, resulting in a final product with a significant growth in the therapeutic field and, as well, to many others useful properties.

### BACKGROUND OF THE INVENTION

Jasmonates compounds are characterized by the cyclopentanone ring and are already known as vegetables hormones produced and delivered in stress situation by vegetables. Qualify as cyclopentanones, the Jasmonates, are potent antibodies in vitro and efficient to reduce tumors cells in vivo, as demonstrated by Flescher et al (US 2002/0173470).

### DETAIL DESCRIPTION OF INVENTION

The Jasmonate family is defined as: methyl jasmonate, jasmonate acid, 7-iso-jasmonate acid, 9,10-dihydrojasmonate acid, 2,3-dihydrojasmonate acid, 3,4-dihydrojasmonate acid, 3,7-dihydrojasmonate add, 4,5-dihydrojasmonate acid, dihydro-7-isojasmonate acid, cur cubic acid, 6-epy-curcubic acid lactones, 12-dihydrojasmonic acid, 12-dihydrojasmonic acid lactones, 11-hydrojasmonic acid, 8-hydrojasmonic acid, homojasmonic add, dyhomojasmonic acid, 11-hydroxi-dyhomojasmonic add, 8-hydroxi-dyhomojasmonic acid, tuberonic acid, tuberonic-0-glucopyranosidic acid, 5,6-dihydrojasmonic acid, 6,7-dihydrojasmonic acid, 7,8-dihydrojasmonic acid, cis-jasmonic, dihydrojasmonic, methylhydrojasmonic, conjugated jasmonic acids with amino acids and esters with lower range alkyl chains linked with all possible substituents and stereoisomer. Optionally the Jasmonate family compounds can be associated as pro drug through an amide and or esters chains, and or with others. The definition of pro drug concerns upon some structures after metabolized inside the chemical environment of an animal body, reactions such as hydrolysis or oxidation/reduction, or any metabolic or catabolic organics reactions, sometimes broken a specific chain and produce two or more others with metabolic, or tabolic action as medical drugs and others. Specifically in this invention this kind of combination could be useful to get a better performance of the final product using A. Since these drugs are encapsulated in the case of the present invention, the Jasmonates family members, as well, its derivates aspects concern to oily and low solubility, can be turned into a soluble molecule in which it will allow a better pharmacokinetics to produce products that can be made as oral, intra dermal, dermal, surgery, topic such as epidermic and mucosas uses, skin appendages, endoscopic procedures as well intra orifices uses, mechanical or guided, laparoscopic procedures, parenteral nutrition, intra brain procedures, lumbar punctures, cosmetically procedures, sub dermal procedure, any tissues procedures, transdermal, spine punctures or procedures, intramuscular, inhalation, ocular, dental, as endogenous administrations, sublingual, subcutaneous, rectal use, or any other uses via mucosas. Also the nano carried, and or, micro carried elements of the Jasmonate family can be modified in its structure to improve his actions towards innumerous different aims. It can be changed in its cyclopentanone ring, increasing or making substitutions, turning it to cyclopentenone, or adding innumerous other elements to its structure to improve its effects. The Jasmonates family elements can also be used to formulate new compound to be included with it inside of the nano, and or, microcarriers.

The nomination micro or nanoparticles applies the control delivery of molecules are enlarge and refers to all types of different structures that are able to form nano spheres, nano capsules, microsphere and micro capsules carriers that can carry de molecules referred in this invention. Denominate are nanospheres are systems that the active principle are homogeneous disperse or soluble inside of the polymeric matrix. In this sense the system obtained are unique and is impossible to distinguish between the host and guest molecules. Otherwise, nanocapsules are systems that are possible to identify the two phase compounds. In these compounds the active principle are possible to distinguish a difference between the two systems, host and guest. Sometimes the two systems are made in different phases, solid and liquid phases. In these cases the substances are involved with polymeric matrix, usually one membrane, isolated to the nucleus.

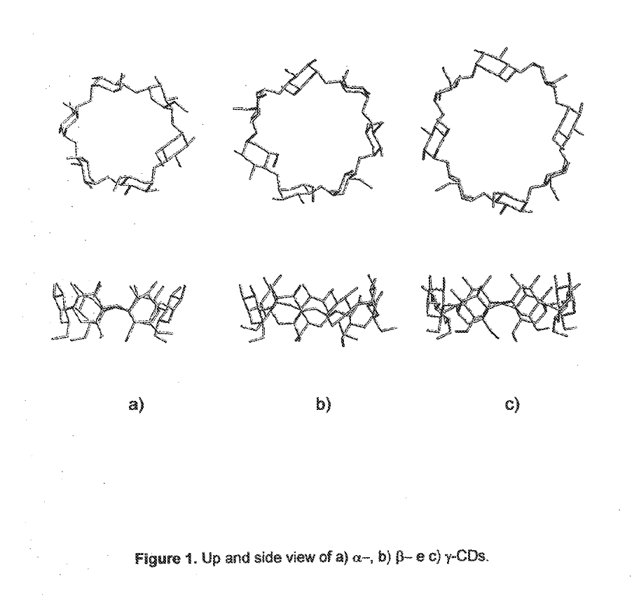

Cyclodextrins (CDs) are cyclic oligosaccharides formed by D-L(+)-Glucose units linked by a-1,4-C—O—C chains. CDs are obtained by the enzymatic degradation of starch with the CGTase glucotransferase. The native CDs are defined by the number of glucose units, α-, β- and γ-CDs obtained with 6, 7 and 8 glucose units. The FIG. 1 shows the molecular structure of these natives CDs.

From the structural point of view the CDs are cone shaped molecules. In the molecular structure of CDs we have two sides, hydrophobic and hydrophilic. Inside of the cavity of CDs one hydrophobic character is predominant. This characteristic is important to guide the guest molecule to locate spontaneously inside the cavity. That principium is called inclusion compounds formation phenomena. In thermodynamic aspects the spontaneous formation rule that the lower energy of a systems are the most probably to occurs. Like the classic experimental formation of micelles. The inclusion compounds formation occurs because it is the lower energy state between the molecules with hydrophobic effect. Otherwise, the hydrophilic part, outside CDs cavity, contributes to the stability of the inclusion compounds formed. This phenomena possibility the use of low soluble or high toxic molecules as active principle as pharmaceutical products. Nowadays this type of technology is already established and useful in pharmaceutical industries and others technique applications.

The CDs can forms inclusion compounds with a notable numbers of guest molecules and are in use with different applications in pharmaceutical, food and cosmetics products. The molecular encapsulation shows practical advantages to news formulations of oldest products. Many of known molecules that were negligence by the industry in the past can be studied in news formulations with these microcarrier and or nanocarries and is very probably that a new product remains a large number of applications. We have mentioned CDs, but there many others elements and molecules, natural, synthetic, semi-synthetic and or mixture that has being projected to build nanocarriers to be able to carry drugs and many others substances inside of it. Each one has a specific aim or property to show its effect. In this patent we are predicting all the micro, and or nanocarrier made of these elements and compounds molecule such as microcarriers, and or, nano carries made of any chemical reaction, using elements naturals or not, semi synthetic materials or not, synthetic materials or not, compounds that will create news formulations of micro or nano capsules or carriers within Jasmonate family members, in its pure formula, and or mixture, conjugated with any other drug or drugs or substance in which may increases its effects, Jasmonate family members with its structure modified in any part along its structure, pure or associated with any other substance derivate from plant or any animal, including elements and substrate from microorganisms, and or, drugs, and or, any active principal to reduce the toxicology of the drug and active molecule having the Jasmonate as a co-factor or a co-helper, or to have the other drug or organic molecules, organic substrates, organic elements, pure, mixture or conjugate, and or, mineral elements, and or synthetic, and or semi-synthetic to improve acting as co-helper, or increasing the active principal of the Jasmonate family members, and or, elements derive from it. To be useful, as an example: In the medical field, in its all areas, to make the formulated molecule reach the aimed target, using any possible components to build up the micro, and or, nano capsules, or carriers, and or biomarkers, that will increasing the action of the Jasmonate's elements and family, and or, its derivate members in conjugation to any other molecules to be increased with any other elements and molecules, proteins, glycoprotein, lipids, organic elements, mineral elements, as single or mull structures compounds molecules such as to be used, as well, at the fields: chemical, physics, mechanical, structure, agricultural, veterinarian, cosmetics, production of elements in the manufacturing products of any kind of industrial fields. Its predict at this patent, all the Jasmonate family members, or derivate from it, as well all the possible molecule formed with it, in its pure form, modified, conjugated, mixture, complexed, or any other molecule with its participating, including in elements or molecules able to be form as an micro, and or, nanocarrier compounds with properties to develop better effect of Jasmonate family members, or derivate from it, with all the possible molecules formed with it, as: any element that has one, or more mixture properties such as, magnet properties, electrical properties, chemical properties, photo sensibility properties, morphologic properties, bio acceptance properties, non rejection properties, physiologic properties, body response properties, protection properties, dental properties, organic, and or not organic ataxia effect, all types of ataxia properties, radiation properties, remote controlled guidance properties to the micro, and or, nano host, fluorescence properties, thermal properties, physics projected new structure for better carrier compounds, also includes modified morphological surface polymers added or conjugated, with pure or synthetic or modified, or mixture with organic components, added or conjugated with pure or synthetic or modified, or mixture lipid components, added or conjugated with pure or synthetic or modified, or mixture mineral components, added or conjugated with pure or synthetic or modified, or mixture metal components, added or conjugated with pure or synthetic or modified, or mixture carbon components, added or conjugated with pure or synthetic or modified, or mixture all elements above with computerized components, added or conjugated with pure or synthetic or modified, or mixture cellular, micro and or nanocarriers made as, within, using parts, added at, into, with, bacteria, or mixtures components, added or conjugated with pure or synthetic or modified, or mixture molecules components and or, virus, or mixtures components, added or conjugated with pure or synthetic or modified, or mixture molecules components and or, fungus, or mixtures components, added or conjugated with pure or synthetic or modified, or mixture molecules components and or, or body solids elements or mixtures components, added or conjugated with pure or synthetic or modified, or mixture molecules components or body's fluids, lymph and blood elements, mixtures components, added or conjugated with pure or synthetic or modified, or mixture molecules components.

Other classes of molecules that can interact forming structures like inclusions as micro and nano carries predict in this present patent are block copolymer as Pluronic, a relative hydrophilic polymer and poli-ε-caprolactone obtained by the broke of the ε-caprolactone ring in presence of PEO-PPO-PEO and catalisator of octane estanoso. (Drumond W. S.; Wang, S. H., 2004). Thea are other biopolymers, or not, that are predicted in this patent that can be used as carriers structure to propose a better effect of the molecule in its expected aim.

The present invention can be a multiple formulation connected upon Jasmonates family members structures and others molecules, and possible compounds, derivate from it, associated with a large number of different elements that forms stable inclusion compounds as nano carries or micro carries, and or nanoemulsions, and others, that can be used to produce all the possibilities mentioned above. As well, the use of this invent in these request patent is to obtain an improvement of the delivery of Jasmonates family members and its derivates molecules, and possible originated compounds, pure, and or, modified, and or, mixture, and or, conjugated and it's various formulations, within, and or, at, and or, with micro and nanocarriers.

The invention also refers to the micro and nano particles within, and or, and or, at Jasmonate originated compounds, and or, pure, and or, modified, and or, mixture, and or, conjugated and it's various formulations, within, and or, at, and or, with, micro and nanocarriers used as the interaction with any drug as members of inhibitory drugs for hypoxic condition at the normal or cancer tissue. Its requett as inventions the interaction of members of the Jasmonate family, and its derivate members and possible molecules with it, included or forming inclusion compounds in micro particles or and into nano particles, as carriers, in its as pure or mixture formula, with members of molecules that can act its effect in the many fundamental biochemical pathways—DNA synthesis, transcription, translation, gene regulation and energy production first developed under conditions of anoxia, as anaerobic glycol sis, that function best in hypoxia and it is these pathways that are up-regulated in the hypoxic cancer cell,

The affect of the invented molecule with it action on the trans-activating domain (TAD) amino acid compositions, which are either essential for Trans activation or are the most abundant amino acids in TAD are used for generation of TADS groups. The Trans activation by transcription factor Gal4 was found to be provided by acidic amino acids and therefore Gal4 is referred to the transcription factors with acidic activation domain. In that order Gcn4 is referred to the transcription factors with hydrophobic activation domain.

Nine-amino-acid trans activation domain (9aaTAD) defines a novel domain common to a large super family of eukaryotic transcription factors represented by Gal4, Oaf1, Leu3, Rf3. Pho4, Gln3, Gcn4 in yeast and by p53, NFAT, NF-κB, any other gene translator factor, nuclear or not, and VP16 in mammals

The affect of the invented molecule with its action on the DNA, RNA of cells, mRNAs, clones, oncogenesis, proto oncogenesis, pro apoptosis cellular's groups, anti apoptosis cellular's groups, anti or pro cellular fadigue, and or senescence, and or virus, and or, fungus, and or bacteria

The affect of the invented molecule with any mentioned action involving immunological cells and its members, and or its substrates. The affect of the invented molecule with all the mentioned invented molecule's action participating of any intracellular interactions, and or, integrations such as the actions mentioned bellow: phosphorylation process, glycolisis, anaerobic, or and, aerobic, energetic metabolic process, involving any mitochondria process, and or the electron transport chain, and or, suppresses or activation of the activity of a group of cysteine proteases called caspases, and or apoptosis inducing factor, and or, Faa receptor (FaaR), and or any group involved death inducing signalling complex_(DISC), and or any function with_adaptor molecule FADD, and or death effector domain (DED) near its amino terminus-which facilitates, binding to the DED of FADD-like ICE (FLICE), more commonly referred to as caspase-8 proteolytic cleavage, and or the effect of the invented molecule, pure or not, conjugated or not, with its actions on, or as, or into the caspase-8 catalyzes the cleavage of the pro-apoptotic BH3-only protein Bid into its truncated form, tBid. Also when all the predict micro and or nano molecules mentioned involved in any circumstances or any mode with BH-3 only members of the Bcl-2 family exclusively engage anti-apoptotic members of the family (Bcl-2, Bcl-xL), any action that may allow the invented molecule to allow or increase the Bak and Bax to translocate to the outer mitochondrial membrane, all process that may act as permeabilizing it and facilitating release of pro-apoptotic proteins such as cytochrome c and Smac/DIABLO, an antagonist of inhibitors of apoptosis proteins (IAPs).

The effect of the invented molecule, pure or not, conjugated or not, with its actions on, or as, involved in any circumstances or any mode with cells of dubbed Type 1 cells that are characterized by the inability of anti-apoptotic members of the Bcl-2 family (namely Bcl-2 and Bcl-xL) to protect from Fas-mediated apoptosis.

When all the molecules formed as predict in this patent is involved in any circumstances or any mode with the characterized Type 1 cells include H9, CH1, SKW8.4 and SW480, all of which are lymphocyte lineages except the latter, which is a colon adenocarcinoma lineage.

The action of the invented molecule, pure or not, conjugated or not, with its actions to the increasing functions, decreasing functions, co-helpers, up regulating, down regulating, acting as an inhibitory factors, activate factor, and or, participating in any metabolic and catabolic cellular action, alone or conjugated, participating as any action as cellular co-factors, at or with all the following intracellular sites: STATS, CR, MAPK, SV 40 promoter-1 (SP1), E26 (Ets), NF-AT, GATA-3, JNKs, Rel A, Rel B, IkBs and all forms of NF-κB, all proteins that belong to the NE-KB complex, AP-1, as agonists or antagonists of the nuclear receptors, all the PPars, to react with lipo-poly sacharides molecules, as substrate or agent, to interact with any agent involved in the inflammatory process, as all cytokines and ICAM-1, VCAM-1, with COX-1, COX-2, as agonists or antagonists of all prostaglandins, leucotrines, pure or not, tromboxans, with all chemo cytokines, monoclonal cells carrying cancer cells, Dendritic cells, T cells, B cells, CD1, CD4, CD8 and any subclass of it, memory cells, natural killers, and all blood cells, blood proteins, pure or not, conjugated or not, natural or artificial cloned cells.

The action of the invented molecule, pure or not, conjugated or not, with its actions to the increasing functions, decreasing functions, co-helpers, up regulating, down regulating, acting as an inhibitory factors, activate factor, and or, of the VEGFs, and all the growing factors, and all its cellular's receptors, as all the others cytokines cellular's receptors, to all metalloproteinases, aFGF, bFGF, its actions on TK cell membrane's receptors, G protein mediated cell membrane's receptors, any other cellular receptor, cell substrates inducers, biomolecule inducers, immunologycal inducers, it's action direct or indirect with PDGF, HIF, polypeptide growth factors, TGFα and TGFβ, (TGFIβ exists in three known subtypes in humans, TGFβ1, TGFβ2, and TGFIβ3), Interferons, Ils, Tumor necrosis factor (TNF, cachexin or cachectin and formally known as tumor necrosis factor-alpha) Lymphotoxin (also known as tumor necrosis factor-beta) and any biological molecule production. All binds cytokines at the following targets: Immunoglobulin (Ig) superfamily, which are ubiquitously present throughout several cells and tissues of the vertebrate body, and share structural homology with immunoglobulins (antibodies), cell adhesion molecules, and even some cytokines. Examples: IL-1 receptor types.

The affect of the invented molecule with any mentioned action involving in any kind, pathway or bonding with Haemopoietic Growth Factor (type 1) family, whose members have certain conserved motifs in their extracellular amino-acid domain. The IL-2 receptor belongs to this chain, whose γ-chain (common to several other cytokines) deficiency is directly responsible for the x-linked form of Severe Combined Immunodeficiency (X-SCID), Interferon (type 2) family, whose members are receptors for IFN β and γ. Tumor necrosis factors (TNF) (type 3) family, whose members share a cysteine-rich common extracellular binding domain, and includes several other non-cytokine ligands like CD40, CD27 and CD30, besides the ligands on which the family is named (TNF).

The affect of the invented molecule with any mentioned action involving in any kind, pathway or bonding with seven, and/or/any transmembrane helix family, the ubiquitous receptor type of the animal kingdom. All G-protein coupled receptors (for hormones and/or neurotransmitters) belong to this family. Chemokine receptors, two of which act as binding proteins for HIV (CXCR4 and CCR5), also belong to this family. all elements belonging and involved at the formation, and or, being produced direct or indirect in the immunological response, involving molecules, and complex substrate of immature and mature dentritics cells,

### Physics and Chemical Characterization of the Invention

As an example, a complete study for natural CDs and Methyl Jasmonate and Jasmonate Acid was performed, which includes all the nano capsules and micro capsules inclusion or process of using the Jasmonates family members, and its derivates carried with it. One of the most popular method, is the characterization of the inclusion compounds formation was theoretical Qualitative Structure Analyses Relationship (QSAR) applied with HYPERCHEM software using semi-empirical approach. In this sense QSAR was used to estimate the stability of the inclusion compounds formed between jasmonic acid and methyl Jasmonate inside of the all natives CDs. The calculation was performed with AM1 semi-empiric method using Polak-Rabiere conjugated gradient with rms of 0.1 kcal (angstron.mol)⁻¹.

Assuming ΔG=ΔH we can write the equilibrium constant of the reaction of formation of the inclusion compounds as a function of entropic term:

ΔG=−RTInK [1]

Where

ΔH=−RTInk [2]

Qualitatively the measurement of ΔH (=E_{binding}) reflex the lowest of the total energy of the system when inclusion compounds formation occurs.

Therefore, the stability of the reaction represented by E_{binding} can be estimated from the total energy of the all individual components of the reaction of formation of the inclusion compounds, as:

S+CD→S:CD [3]

In this way we can write:

ΔH=ΔH_{fs:CD}−(ΔH_{fS}+ΔH_{fCD}) [4]

The Table 1 shows the results of the calculation for the inclusion compounds formation between methyl Jasmonate and jasmonic acid and the natives CDs.

TABLE 1
The result of the ΔH of stabilization.

| | Ebinding (Kcal.mol-1) |
|---|---|
| Jasmonic acid-α-CD | −9.63 |
| Jasmonic acid-β-CD | −19.64 |
| Jasmonic acid-γ-CD | −2.02 |
| Methyl jasmonate-α-CD | 8.44 |
| Methyl jasmonate-β-CD | −31.35 |
| Methyl jasmonate-γ-CD | −18.23 |

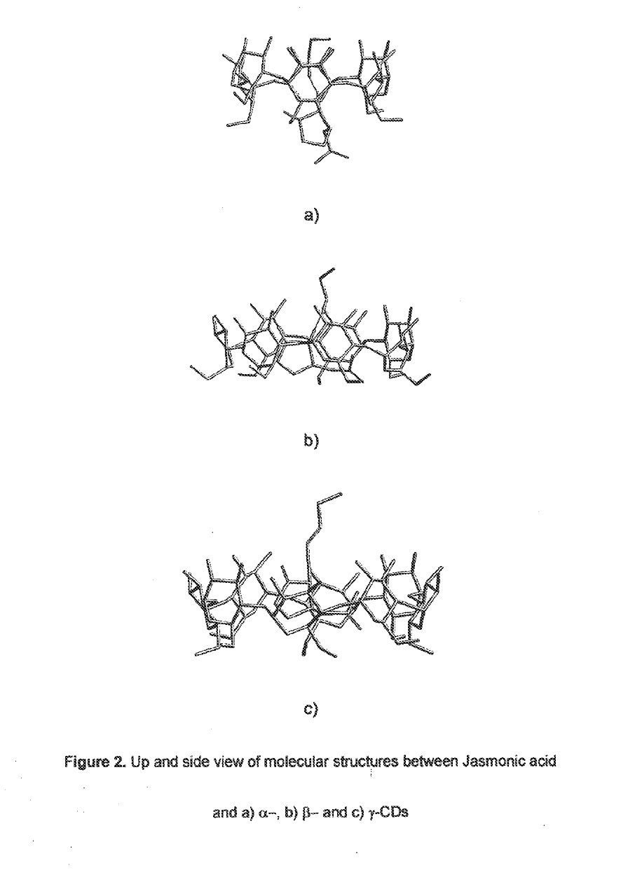

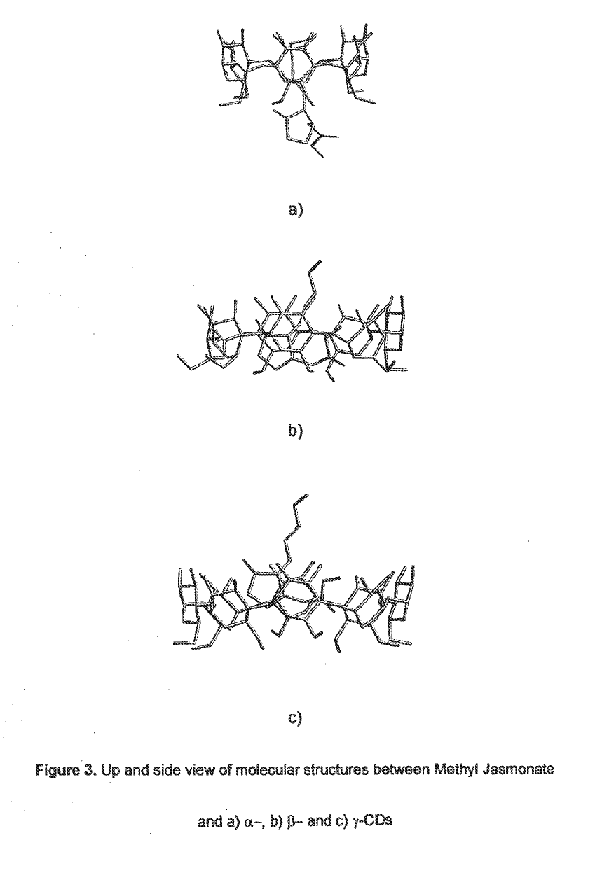

Observing the Table 1 we can distinguish some significant results. First, with exception of the complex between α-CD and methyl Jasmonate, all the inclusion compounds between Jasmonic acid and methyl jasmonate with native CDs are stables. Second, in both cases the complex with β-CD is most stable. Others results is not so clear. With Jasmonic acid α-CD is most stable than γ-CD and with methyl Jasmonate is the reverse. FIG. 2 shows the molecular structure of lower energy for Jasmonic acid and FIG. 3 shows the results for methyl Jasmonate. Note that in the case of α-CD the guest molecules in both cases are a little outside to the cavity than the other CDs.

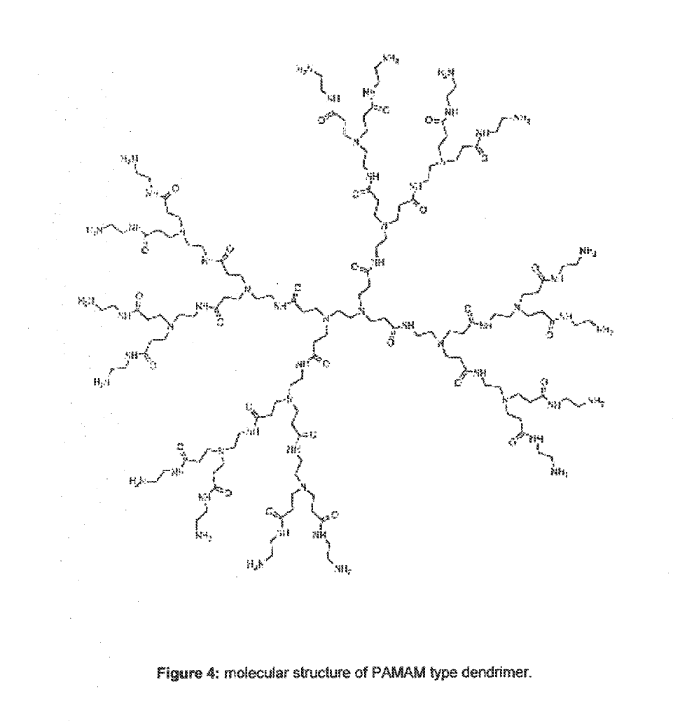

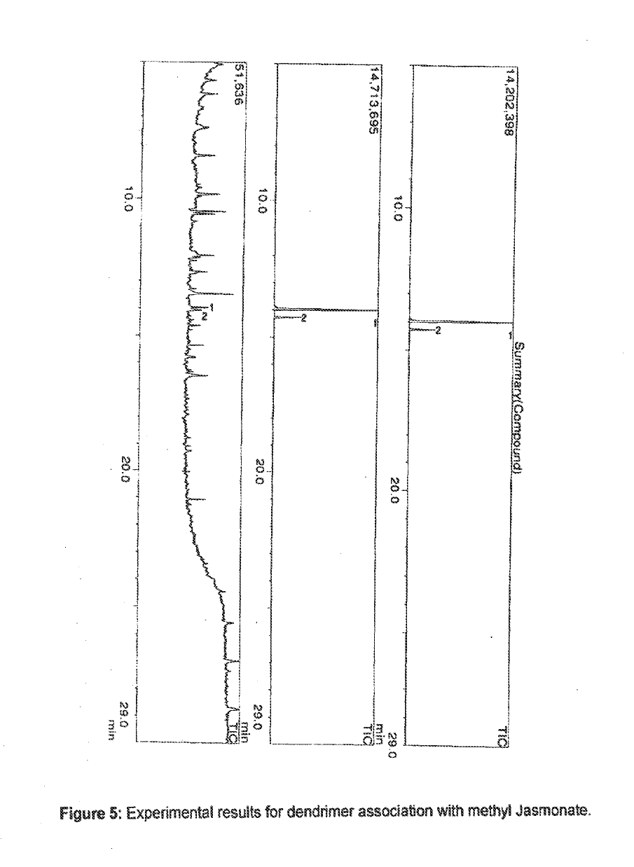

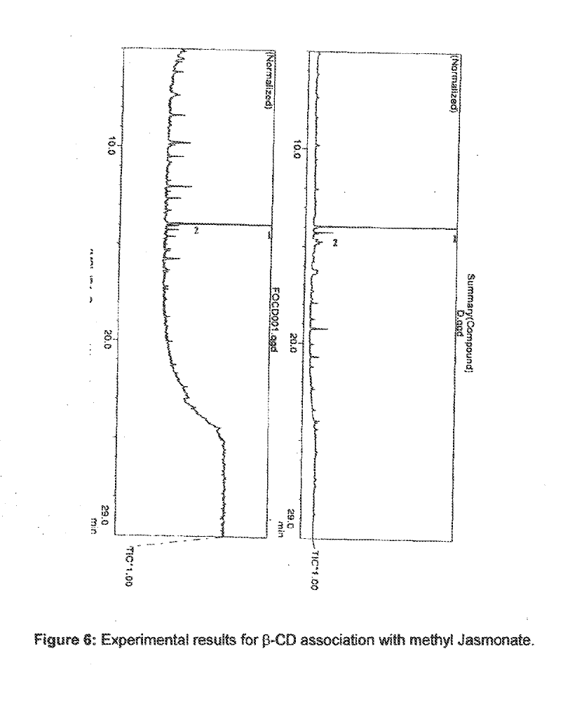

To prove the universal correlation with others nano carries we make experimental analyses with GC/MS equipment for dendrimers and CDs to compare the analytic comportment. Observe that class of dendrimers used was PAMAM. FIG. 4 shows the general structure of PAMAM dendrimers. FIG. 5 shows the results for dendrimers and FIG. 6 shows the results for β-CD, both with methyl Jasmonate.

The experimental data were performed at a GC with column on temperature of 50° C., the injection temperature of 250° C., Flow control mode linear, total flow 50.0 mL/min., column flow of 1.70 mL/min., the percentage relative to the incorporation of methyl jasmonate inside β-CD was 98-99% for dendrimer PAMAM was more than 95%. Comparing the peak 1 with 2, the peak 2 is the methyl Jasmonate alone and peak 1 is the inclusion compounds formed, in both cases the peak 1 is Cleary different and proves de molecular association.

### Examples of the Preparation of Invention

Inclusion compounds between the Jasmonates and nano carries or micro carries can be prepared mixing a concentration different than zero until 1 mol. Molar with equivalent proportion of host molecule. The procedure of preparation can be mixer the Jasmonate family compound at a solution in water or other solution with pharmaceutical acceptable salts. The resulting solution is stirred until total dissolution of the components in the solvent. Usually the time of mixer is some hours at the mixture get the thermodynamic equilibrium (Rajewski & Stella, 1996).

### PATENT REFERENCES

CA 2,630,666
EP 1814894
US 2002/017347

### NON PATENT REFERENCES

Drumond W. S.; Wang, S. H. “Sintese a Caracterizaçáo do copolimero Poli ácido lático-B-Glicol Etil\tilde{e}nico” Polímeros: Ciência e Tecnologia, 14, n 2, p. 74-79, 2004
Rajewski R A, Stella V J, Pharmaceutical applications of cyclodextrins. 2. In vivo drug delivery. J Pharm Sci 1996; 85(11): 1142-69.

1. Pharmaceutical formulation comprising an active principal from Jasmonate's family members and its derivates elements and all the molecule, and/or possible formulations, and/or compounds into, and/or inside, and/or at a nanocarrier or microcarrier, as hosts, that can protect the molecule of Jasmonate family's members and all its derivates from chemical, and/or, any other environmental reactions.

2. Pharmaceutical formulation, how described in claim 1, where the active principle is preference all members from Jasmonates family and its derivates molecules with, and/or into, and/or inside, and/or at a nanocarrier, and/or microcarrier, and/or original, and/or synthetic created, and/or with substitutions, and/or not, and/or in any of its structure's formula, and/or formed through conjugations, and/or any different association, pure, or not, with any kind of organic element, and/or mineral element, and/or synthetic elements, and/or any other substance.

3. Pharmaceutical formulation, how described in claim 1, where the CD used is optionally 2-,4-,6-trismethyl-CD, and/or all heptakis-6-sulphate-CD, and/or all hydroxypropyl-CD, and/or all large ring CDs, with a range of units between up to 8 until 26 glucose units, or chains, and/or polymers, and/or any associations, and or all associated or not with smallest CDs inside of the cavity of large ring CDs forming a double system inclusion compounds, and/or all other denominate CD what can be synthetic create, linked with any kind of organic, and/or all mineral elements, and/or all biological elements, pure, or not, and/or mixture, and/or as pro drug associated with DNA or RNA type of molecules as its claim the Jasmonate family's member and it's derivates, modified by any possible inclusion, and/or conjugations, and/or, by any change at its molecule, or not, within CDS that that are changed chemically, and/or morphologically, pure or not, and/or conjugated or not, and/or to become electric charged, and/or magnified, and/or photo reactive, and/or light emission structures, and/or PH reactive, and/or chemically reactive, and/or radiation sensible and reactive, and/or computerized controlled drug delivery, and/or computerized structure for molecule guidance, and/or to be controlled at a distance, and/or associated at the nano or microcarrier, and/or any substance such as chemotherapy agents, and/or any antibiotic, and/or any antifungal, and/or any anti-inflammatory, and/or any corticoid, and/or any protein, and/or any carbohydrates, and/or any hormones, and/or any lipids and/or any cosmetic, and/or any agriculture issue and/or any industrial uses.

4. Pharmaceutical formulation, how described in claim 1, where the CDs can be alternatively substituted from a list of host molecules comprising liposome, different kinds of dendrimers as a drug carrier independent of its formations and built structures, as an example: PAMAM/MTX and PAMAM-PEG/MTX, and/or other dendrimers types or not, and/or supramolecular nanocarrier for gene and siRNA delivery, and/or semi-permeable polymer nanocarriers for enzyme therapies, and/or all kinds of polymers, and/or all biopolymers or not, and/or all synthetic, or not, and/or all pure, or not, and/or any made as a singular element, or not, and/or composed in chains with more than one types of polymers and biopolymers, and/or mixture or not, and/or with other kinds of nano e microcarriers, and/or including, as well, all nano and also microcarriers having in their structures any kind of elements, as pure, and/or as mixture, and/or as natural, and/or as modified, and/or as semisynthetic, and/or as synthetic, and/or as made of heteromorphism structures, and/or as homomorphism structures, in which includes elements as part of its structure, and/or as the carrier itself such as biological structures, and/or as blood elements as carriers or any other use, and/or as biological and/or colloidal substances, and/or as natural, and/or as mixture, and/or as synthetic, and/or as semi-synthetic, and/or as any proteins, pure or not, and/or natural or not and/or any glycoprotein, pure or not, and/or natural or not and/or enzymes, pure or not, and/or natural or not and/or lipoglycoproteins, pure or not, and/or natural or not and/or antibodies, pure or not, and/or natural or not and/or cells, vegetal cells, pure or not, and/or natural or not and/or organic fluids, pure or not, and/or natural or not and/or elements, pure or not, and/or natural or not and/or animal subtracts, pure or not, and/or natural or not and/or, plants subtracts, pure or not, and/or natural or not and/or vegetal elements and compounds, pure or not, and/or natural or not and/or fungus substrate and/or itself, pure or not, and/or natural or not and/or virus substrate or itself, pure or not, and/or natural or not and/or bacteria substrate or itself, pure or not, and/or natural or not and/or natural compounds and its substrates, pure or not, and/or natural or not and/or crystals, carbon structure, gold or any metal, nano spheres, and/or microspheres mixture or pure, liposome, pure or not, and/or modified, and/or mixture, and/or synthetic, and/or unique lamellar vesicles, and/or multi lamellar vesicles, and/or alternatively administered with polymers artificial, and/or LDE, pure or not, and/or all kinds of polymeric structures, pure or not, and/or synthetic or not and/or all kinds of dendrimers structures, pure or not, and/or synthetic or not and/or talospheres structures, pure or not, and/or synthetic or not and/or nano spheres structures, pure or not, and/or synthetic or not and/or metal structures, pure or not, and/or synthetic or not and/or mechanical structures, pure or not, and/or synthetic or not and/or computerized structure, pure or not, and/or synthetic or not and/or electronic structure, pure or not, and/or synthetic or not and/or magnetic structure, pure or not, and/or synthetic or not and/or biological made, pure or not, and/or synthetic or not and/or or biological sensible structure, pure or not, and/or synthetic or not and/or chemical sensible structure, pure or not, and/or synthetic or not and/or radiation sensible structure, pure or not, and/or synthetic or not and/or thermal sensible structure, pure or not, and/or synthetic or not and/or electric sensible structure, pure or not, and/or synthetic or not and/or biopolymers, pure or not, and/or synthetic or not and/or mixture or pure, mixtures lipo-polymers, pure or not, and/or synthetic or not and/or organic substances able to become a micro, and or a nanocarrier of the Jasmonates family member compounds, mixture with all kinds of elements pure or not, and/or synthetic or not and/or nano particles made of pure or mixture with mineral elements, such as silicon, pure or not, and/or synthetic or not and/or silicon, pure or not, and/or synthetic or not and/or carbon, pure or not, and/or synthetic or not and/or, all kinds of dendrimers, pure or mixture, simple or combined” with other elements, or micros spheres, or micro carries in general or polymeric CDs, or CDs nano containers (carcerands), or crystals or metals nanostructure, pure or not, and/or synthetic or not and/or any other biological structure, pure or not, and/or synthetic or not and/or, mineral structure, mixture or pure, and/or synthetic structure mixture or pure, and/or others modified at the surface, and/or within inductive substances, and/or with the following effects, blocking, and/or conduction, and/or producing, and/or patenting, and/or specially of patenting biological tropism, and/or analogues, and/or competitive, and/or synergic, and/or superficial modified, and/or with multi inductive substances, and/or interactive, and/or lighting nanoparticles, and/or nanoparticles derived from silica, and/or silicon, evaporating particles, and/or heat sensible particles, and/or gas and/or any other chemical liberation particles, micro and/or nanoparticles formed as target or specific receptors, and/or at vegetables, and/or animals and/or yours derivatives, optical and/or perfusion fluids with the use of preservation of organic tissues, and/or transplant, and/or any other tissue use, and/or nanocarrier systems to enhance the bioavailability of drugs at the disease site, nanocarrier system incorporated with any stimuli-responsive property developing as its deliver, or not, due any physics, and/or chemical reactions (e.g., pH, temperature, or redox potential, Temperature, pH, and hypoxia are examples of “triggers” at the diseased site that could be exploited with stimuli-responsive nanocarriers), and/or any other response, involving itself only, and/or, other molecules, and/or substances, including stimuli-responsive nanocarrier systems for drug and gene delivery, and/or any nanocarrier able to perform the effect of gene delivery, carriers made that can respond to biological stimuli, and/or microcarriers, and/or nanocarriers able to chance it s morphologic structure during its delivering procedure, and/or, before it, and or after it such as the optimization of nanocarrier size and surface charge modulation.

5. Pharmaceutical formulation, how described in claim 1, to use in chemical therapeutic treatment for cancer to use in chemical therapeutic treatment for cancer in humans in the sense to decrease the side effects, in association with any kind of treatment, as a single therapy, and/or mixture therapies, and/or with its actions on the interference in cell growth, and/or, inhibited signal, and/or, the interference in cell's apoptosis, and/or, the effect of the claim molecules be used, partially or in its total, and/or as an ingredient, and/or, an element, and/or part of any compound, with its action towards cell's, such as in the effect of the limitless cell's replicative potential, and/or in the sustained angiogenesis by cell, and/or in favor effect of antiangiogenisis, and/or cell's controlling tissue invasions and/or cancer metastasis, and/or any effect at the tumors development, and/or its regressions, and/or the use of this molecule in any other therapy such as: biotherapy, and/or chemotherapy, and/or radiotherapy, and/or angiogenic, and/or antiangiogenic therapy, and/or genetic therapy, and/or surgeries, and/or bio molecular manipulation as part of any therapy, and/or surgery of any kind, and/or plastic surgery, and/or photo dynamic therapy procedures, and/or dental procedures, and/or cosmetic surgery, and/or any cosmetically appliance, and/or any surgery healing scars and wounds, and/or with any kind of laser and light therapy, and/or to be used as drugs for AIDS and/or any virus disease, and/or bacterial disease, and/or body dysfunctions, and/or as a component to be used as anti-inflammatory active, and/or pain reliever, and/or anti-septic solutions, and/or anti-fungus solutions, and/or anti-virus solutions, and/or anti-bacteria solutions, and/or be part of any chemical purpose, and/or as drug for fungal disease, and/or auto immune disease, and/or dystrophies, and/or mental diseases, and/or depressions, and/or with, associations with neurological effects, and/or antidote for poisoning or any other dangerous or harm affections, and/or inducer or activator of mechanisms for bioresults, and/or as anti-smoking drugs, and/or vaccines, and/or being any other kind of bodies intracellular, and/or extracellular activator, and/or, body's and/or any organic molecules as an inhibition factors, and/or angiogenic or antiangiogenic therapies, single or in an associations therapy in animals, and/or in humans in the sense to decrease the side effects of any kind.

6. Pharmaceutical formulation, how described in claim 5, wherein the drugs are encapsulated, such that the Jasmonates family members, as well, its derivates aspects concern to oily and low solubility, can be turned into a soluble molecule in which it will allow a better pharmacokinetics to produce products that can be made as oral, and/or intra dermal, and/or dermal, and/or surgery, and/or topic such as epidermic and mucosas uses, and/or skin appendages, and/or endoscopic procedures as well intra orifices uses, and/or mechanical or guided, and/or laparoscopic procedures, and/or parenteral nutrition, and/or intra brain procedures, and/or lumbar punctures, and/or cosmetically procedures, and/or sub dermal procedure, and/or any tissues procedures, and/or transdermal, and/or spine punctures or procedures, and/or intramuscular, and/or inhalation, and/or ocular, and/or dental, and/or as endogenous administrations, and/or sublingual, and/or subcutaneous, and/or rectal use, and/or any other uses into mucosal, and/or at, and/or inside.
